(19) United States
(12) Patent Application Publication
(10) Pub. No.: US 2014/0378405 A1
(43) Pub. Date: Dec. 25, 2014

(54) SALIX EXTRACT, ITS USE AND FORMULATIONS CONTAINING IT

(71) Applicants: Ezio BOMBARDELLI, Groppello Cairoli (IT); Andrea GIORI, Milano (IT); Alessandro ANELLI, Milano (IT)

(72) Inventors: Ezio BOMBARDELLI, Groppello Cairoli (IT); Andrea GIORI, Milano (IT); Alessandro ANELLI, Milano (IT)

(21) Appl. No.: 13/925,393

(22) Filed: Jun. 24, 2013

(51) Int. Cl. A61K 31/7028; A61K 31/352; A61K 36/76

(52) U.S. Cl. CPC A61K 31/7028; A61K 36/76; A61K 31/352
USPC 514/35

(57) ABSTRACT

The present invention relates to an extract of Salix spp obtainable by fractioning on a resin and to the process for its preparation.

The extract of the invention is characterized by an high content in salicin derivatives, reduced content in high molecular tannins and a content in proanthocyanidins sufficient to inhibit some tissue metal proteases. The product is formulated in oils rich in ω-3 and ω-6 acids which provide a better absorption of the extract active principles, also increasing synergetically their action.

### SUMMARY OF THE INVENTION

The present invention relates to an extract of Salix spp obtainable by fractioning on a resin and to the process for its preparation.

The extract of the invention is characterized by a high content in salicin derivatives, a reduced content in high molecular tannins and a content in proanthocyanidins sufficient to inhibit some tissue metal proteases. The product is formulated in oils rich in ω-3 and ω-6 acids which provide a better absorption of the active principles, also increasing synergistically their action.

### TECHNOLOGICAL BACKGROUND

Bark and branches extracts of different species of Salix have been used for unmemorable time for the treatment of articular rheumatic forms and gout. Salix extracts were, however, substantially abandoned at the end of the 19^{th} century, when aspirin was synthesized by acetylation of salicylic acid, obtained by oxidation of the compounds present in Salix. However, aspirin and Salix extracts have substantial differences in terms of mechanisms of action and activity on bone joints. The extracts act on the enzyme COX 2, while aspirin mainly acts on COX 1, which involves the well-known side effects on the gastrointestinal tract and blood coagulation, which severely restrict its prolonged use which is conversely necessary in the case of such chronic-degenerative pathologies as arthrosis and rheumatoid arthritis.

It is known from the literature that Salix extracts have extremely variable contents in salicin derivatives, which are on the average up to 15%, and tannin content ranging from 8 to 20%. Tannins present in Salix extracts, as is the case with all gallic and catechic tannins, have strong affinity with proteins and proteoglycans, which involves tissue damages in case of long-lasting treatments.

There is therefore the need for a convenient process, which is easily applicable in the industry and provides extracts with standardized contents in the active components.

### DISCLOSURE OF THE INVENTION

The present invention relates to a process for the preparation of novel Salix spp extracts, characterized by an high content in salicin derivatives, a reduced content in high molecular tannins and a sufficient content in proanthocyanidins in order to inhibit some tissue metal proteases.

It has surprisingly been found that the extraction of Salix bark or branches under suitable conditions, and the specific purification treatment of the resulting extract, provides extracts with salicin derivatives content up to 50%, tannin content not above than 5% and oligomeric procyanindins content higher than 5%.

The advantage of using Salix extracts, particularly the extracts of the present invention, compared with salicin derivatives alone, is connected to the presence of proanthocyanidins, strong radicals scavengers and powerful inhibitors of metal proteases, which are activated in arthritic conditions via over-expression of leukocyte I1₁.

The process of the invention for the preparation of Salix extracts differs from those of the prior art in the extract contents in salicin and its derivatives and in the use of matrices which provide the selective reduction in tannin contents, while retaining the therapeutically useful proanthocyanidins in the extract.

The process of the invention includes four main steps:
a) Extraction of Salix spp branches and bark with suitable solvents which solubilize the desired products (total extract);
b) Removal of water-insoluble (or poorly soluble) tannins;
c) Removal of water-soluble tannins;
d) Increase of salicin derivatives through a purification on an adsorption resin column.

Step (a) is accomplished by extraction of the vegetable material, consisting of plant bark and branches, with a C1-C3 alcohol or acetone or mixtures of these solvents or aqueous solutions of these solvents or water alone. A 30% v/v water-ethanol solution is preferred.

The extraction temperature can range from 10° C. to 80° C., preferably is 25° C.

Step (b) allows to remove water-insolubles, particularly high molecular tannins, from the extract.

Step (c) allows to remove most water-soluble tannins from the extract. This is an optional step, that can be carried out to remove any tannins still present in the extract after step (b). These metabolites can be removed by using polyvinylpolypyrrolidone (PVPP).

Step (d) allows to fractionate the extract removing most useless metabolites (sugars, and the like), while keeping the desired secondary ones, i.e. salicin derivatives and oligomeric proanthocyanidins. This step consist in a chromatographic separation through adsorption on a polymeric resin. Examples of suitable resins for this purpose are Styrene-DVB resins such as AmberliteHP20® or Rohm and Haas XAD1180®, and acrylic resins such as Rohm and Haas XAD7HP®.

During the column fractionation step using suitable solvent mixtures, free salicin can be separated from its derivatives, obtaining fractions rich in free salicin with reduced amounts of its derivatives and fractions with completely different compositions.

The total extract obtained from Salix bark and branches with 30% ethanol is concentrated to a dry residue ranging from 5% to 50% w/w, preferably 25% w/w, and left at a temperature from 1° C. to 20° C., preferably at 4° C., without stirring for a time from 1 hour to 24 hours, preferably 16 hours.

The resulting suspension is centrifuged at 4° C. to remove the residual precipitate containing high molecular derivatives and tannins from the clear aqueous solution.

Water-insoluble or poorly soluble tannins contained in the total extract are removed by water purification, which can be further improved by optional treatment (step c) with polyvinylpolypyrrolidone (PVPP).The partial water purification (step b) can only remove part of tannins (above 50% w/w. of tannins present), while PVPP purification removes residual tannins within values below 5% of the final extract weight.

Therefore, the clear aqueous solution from step b) is treated with PVPP (1-50% w/w, preferably 1:30, most preferably 1:20 on the dry residue of the aqueous extract to treat) keeping stirring for 1 or more hours.

The solution is filtered from PVPP and adsorbed tannins. Then the aqueous solution is adsorbed on the resin, thoroughly washing the substrate with water to remove undesired soluble substances. The solution unretained is discarded.

The product is eluted with a water-alcohol solution (C1-C3 alcohols, preferably ethanol), with water content ranging from 50% v/v to 0% v/v, preferably 10% v/v. Alternatively, a water-acetone solution with water content ranging from 50% v/v to 0% v/v, preferably 10% v/v, can also be used. The water-ethanol solution is concentrated to dryness or atomized. The resulting extract can be formulated in the ordinary pharmaceutical solid forms or as an oily suspension in capsules, particularly in oils rich in ω-3/ω-6 poly-unsaturated fatty acids; particularly preferred are Enothera biennis oil and fish oil and its derivatives.

Active dosages for the treatment of arthrosis and rheumatoid arthritis in humans range from 100 to 1000 mg daily, according to the severity of the disease to treat.

The invention is described in greater detail in the following examples.

### EXAMPLE 1

### Extraction of Salix Branches and Bark with a Water-Ethanol Solution (Step a)

In this step, the total extract which can be used as the starting material for the subsequent column chromatography separation is prepared.

1000 grams of Salix branches and bark are covered with 1.5 liters of 30% v/v ethanol at 20° C. for 4 hours in a static percolator. After 4 hours, the percolate is recovered and extracted 6 times again under the same conditions, but using 1 liter of solvent per extraction, to obtain approx. 7 liters of total percolate. The combined percolates are filtered to remove impurities and vegetable residues. This solution (product 1) has a total dry residue of 154 grams, the yield vs. starting material being 15.4% w/w.

The free salicin HPLC content is 4.63%; the total salicin HPLC content is 15.4% w/w. The tannin content is 16.26% w/w.

### EXAMPLE 2

### Extraction of Salix Branches and Bark with a Water-Acetone Solution (Step a)

In this step, the total extract which can be used as the starting material for the subsequent column chromatography separation is prepared.

1000 grams of Salix branches and bark are extracted with 1.5 liters of 80% v/v acetone at 20° C. for 4 hours in a static percolator. After 4 hours, the percolate is recovered and extracted 6 times again under the same conditions, but using 1 liter of solvent per extraction, to obtain approx. 7 liters of total percolate. The combined percolates are hot filtered and concentrated by a rotary evaporator at 60° C. under reduced pressure. This extract has a total dry residue of 143 grams, the yield vs. starting material being 14.3% w/w.

The free salicin HPLC content is 4.3%; the total salicin HPLC content is 15.7% w/w. The tannin content is 15.42% w/w.

### EXAMPLE 3

### Extraction of Salix Branches and Bark with Water (Step a)

In this step, the total extract which can be used as the starting material for the subsequent column chromatography separation is prepared.

1000 grams of Salix small branches and bark are covered with 1.5 liters of water at 20° C. for 4 hours in a static percolator. After 4 hours, the percolate is recovered and extracted 6 times again under the same conditions, but using 1 liter of solvent per extraction, to obtain approx. 7 liters of total percolate. The combined percolates are filtered with suction and concentrated by a rotary evaporator at 60° C. under reduced pressure. This extract has a total dry residue of 167 grams, the yield vs. starting material being 16.7% w/w.

The free salicin HPLC content is 3.94%; the total salicin HPLC content is 13.6% w/w. The tannin content is 6.8% w/w.

### EXAMPLE 4

### Extraction of Salix Branches and Bark with Methanol (Step a)

In this step, the total extract which can be used as the starting material for the subsequent column chromatography separation is prepared.

1000 grams of Salix branches are covered with 1.5 liters of methanol at 20° C. for 4 hours in a static percolator. After 4 hours, the percolate is recovered and extracted 6 times again under the same conditions, but using 1 liter of solvent per extraction, to obtain approx. 7 liters of total percolate. The combined percolates are filtered and concentrated by a rotary evaporator at 60° C. under reduced pressure. This extract has a total dry residue of 101 grams, the yield vs. starting material being 10.1% w/w.

The free salicin HPLC content is 5.9%; the total salicin HPLC content is 19.9% w/w. The tannin content is 14.5% w/w.

### EXAMPLE 5

### Purification of the Extract of Salix Branches and Bark (Step b): Removal of Water-Insolubles

Solution 1 obtained at the end of the workup described in Example 1 (step a) is concentrated by a rotary evaporator at 60° C. under reduced pressure, to obtain an aqueous suspension with a dry residue of 25% w/w of the total aqueous suspension, the total weight of said solution being 615 g.

The resulting aqueous suspension is cooled at 4° C. and left to stand for 16 hours, then the still cold aqueous suspension is centrifuged at 3000 g for 20 minutes to separate the precipitated residue from the clear aqueous solution. This precipitate, having a dry residue of 16.3 g, is rich in tannins and high molecular products and is removed.

The resulting clear solution (solution 2) has a dry residue equivalent to 137 g of partially purified extract having HPLC content in free salicin of 5.0% and HPLC content in total salicin of 16.7% w/w. The tannin content is 6.9% w/w.

The weight yield vs. starting material is 13.7% w/w.

### EXAMPLE 6

### Purification of the Extract of Salix Branches and Bark (Step c)

The clear aqueous solution obtained at the end of the partial purification process of step b (Example 5, solution 2), having a dry residue of 137 g, is treated to remove water-soluble tannins.

The solution is added with 14 g of PVPP, corresponding to approx. 10% w/w. of the dry residue of the extract to treat. After stirring for 1 hour at room temperature, PVPP is separated from the solution by centrifugation.

The resulting solution (solution 3) has a dry residue equivalent to 125 g of partially purified extract, having HPLC content in free salicin of 5.3% and HPLC content in total salicin of 8% w/w. The tannin content is 1.2% w/w.

The weight yield vs. starting material is 12.5% w/w.

### EXAMPLE 7

### Chromatographic Purification of the Extract of Salix Branches and Bark (Step d)

The aqueous solution obtained from step c (Example 6, solution 3) is loaded onto a chromatographic column containing 1250 ml of Rohm and Haas XAD1180® resin conditioned with water. The water-alcohol solution is adsorbed to the resin, while the unretained solution exiting the column is discarded. The resin is then washed with 1.25 liters of water, removing also this solution as its content in desired components is negligible. These discarded aqueous solutions (product 1) have in fact a total dry residue of 52.6 g, with HPLC content in free salicin of 0.87%, and HPLC content in total salicin of 0.92% w/w.

The column is eluted with 3.75 liters of 90% v/v aqueous ethanol. The resulting eluate is recovered and dried at 60° C. under reduced pressure, to yield 72.4 grams of dry product (product 2), corresponding to a yield vs. the starting material of 7.2% w/w. HPLC content in free salicin is 8.95%, total salicin HPLC content is 30.0% w/w. The content in oligomeric proanthocyanidins is 11.2% w/w, the content in tannins is 2.1% w/w.

### EXAMPLE 8

### Chromatographic Purification of the Extract of Salix Branches and Bark (Step d) and Separation of Free Salicin from its Derivatives

The aqueous solution from step c (Example 6, solution 3) is loaded onto a chromatographic column containing 1250 ml of Rohm and Haas XAD1180® resin conditioned with water. The water-alcohol solution is adsorbed to the resin, while the unretained solution exiting the column is discarded. The resin is then washed with 1.25 liters of water, removing also this solution as its content in desired components is negligible. These discarded aqueous solutions (product 3) have in fact a total dry residue of 51.7 g, with free salicin HPLC content of 1.34%, and total salicin HPLC content of 1.41% w/w.

The resin is washed with 2.5 liters of 10% v/v aqueous ethanol, to obtain a solution (product 4) with a dry residue of 17.1 g (yield vs. starting material of 1.7% w/w.). Free salicin HPLC content is 34.6%, total salicin HPLC content is 34.9% w/w.

The column is eluted with 3.75 liters of 90% v/v aqueous ethanol. The resulting eluate is recovered and dried at 60° C. under reduced pressure, to yield 43.2 grams of dry product (product 5), corresponding to a weight yield vs. starting material of 4.3% w/w, HPLC content in free salicin of 0.45%, HPLC content in total salicin of 39.7% w/w.

### EXAMPLE 9

### Formulation of the Extract in Soft-Gelatin Capsules

Formulation of Salix rubra extract in oily suspension for soft-gelatin capsules.

### Unit Composition

| | | | |
|---|---|---|---|
| Salix rubra extract according to Example 7 | | 250 | mg |
| Gliceryl monostearate | | 30 | mg |
| Soy lecithin | | 10 | mg |
| Enothera biennis oil | q.s. to | 700 | mg |

### Preparation

1) Heat Enothera biennis oil at about 70° C. and melt glyceryl monostearate therein, under stirring.

2) Add soy lecithin to the resulting solution.

3) Disperse Salix rubra extract in the resulting solution, promoting the homogeneous distribution with a suitable stirring system.

Gradually cool the resulting solution under stirring.

1.-9. (canceled)

10. A process for the preparation of extract with salicin derivatives content up to 50%, tannin content not higher than 5% and oligomeric procyanindins content higher than 5%, comprising the steps of:
a) extraction of Salix spp branches and bark with suitable solvents which solubilize a total extract;
b) removal of water-insoluble or poorly soluble from the total extract tannins by using polyvinylpolypyrrolidone (PVPP);
c) removal of water-soluble tannins; and
d) purification on an adsorption resin column to increase the content in salicin derivatives.

11. (canceled)

12. (canceled)
